(19) United States
(12) Patent Application Publication
(10) Pub. No.: US 2023/0190969 A1
(43) Pub. Date: Jun. 22, 2023

(54) METHOD, SYSTEM AND DEVICE TO REDUCE UNWANTED CELLS IN BIOLOGIC PRODUCTS

(71) Applicant: Nickolas Romcevich, Longmont, CO (US)

(72) Inventor: Nickolas Romcevich, Longmont, CO (US)

(21) Appl. No.: 18/047,463

(22) Filed: Oct. 18, 2022

Related U.S. Application Data
(60) Provisional application No. 63/256,905, filed on Oct. 18, 2021.

(51) Int. Cl. A61L 2/00; A01N 1/02

(52) U.S. Cl. CPC A61L 2/0011; A01N 1/0284; A61L 2202/21; A61L 2202/22; A61L 2202/23

(57) ABSTRACT

The disclosure is a medical device system that applies pressure to biologic products in a container to inactivate pathogens (e.g., viruses, bacteria, including sepsis, fungi, parasites, prions, mold and other harmful microorganisms), or abnormal or damaged cells (e.g., cancer, carcinoma in situ, lipoproteins, lipids, antibodies), while preserving the desired cells and the efficacy of the biologic product (e.g., whole blood, plasma, red blood cells, platelets and cells derived from blood, bone marrow, stem cells, brain dura matter, bone graft, skin graft or other bodily sources, either allogeneic or autologous). The biologic product may be autologously sourced then processed and transfused or administered, or be allogeneicly sourced and then processed and transfused or administered.

### RELATED APPLICATIONS

This application claims priority to U.S. Provisional application 63/256,905,titled “Method, System and Device to Reduce Unwanted Cells in Biologic Products” filed Oct. 18, 2021 the entire contents of which are hereby incorporated by reference in its entirety.

### BACKGROUND

High pressure processing (HPP) is a cold pasteurization technique which consists of subjecting product in a sealed, flexible and water-resistant packaging, to a high level of hydrostatic pressure (pressure transmitted by a liquid) up to 600 MPa/87,000 psi for a few seconds to a few minutes.¹ ¹ https://www.hiperbaric.com/en/hpp

Researchers have performed preliminary analysis of high pressure processing to reduce the pathogen load on blood and blood components, with encouraging conclusions. Bradley et al. found promising results of virus inactivation in plasma while maintaining plasma functionality. The research, using a custom-built apparatus, concluded that, “high-pressure procedures may be useful for the inactivation of viruses in blood and other protein-containing components.” They chose a Lambda phage as the model system, “because it is small, has no plasma membrane, and thus should serve as a model system for small, nonencapsulated viruses, which are the most difficult to inactivate by current procedures.” Accordingly, this shows promise for high pressure processing to inactivate nonencapsulated viruses, such as parvovirus B19 and hepatitis A virus, as well as “encapsulated viruses (hepatitis B virus and HIV) and larger organisms such as bacteria and parasites.”

Similarly, Yang, et al.² found that a cycle at 250 Mpa preserved the plasma while inactivating the majority of pathogens, in line with the Chinese FDA's guidance. Under the conditions of 200-250 MPa with 5×1 minute multi-pulsed high pressure at near 0° C., “the inactivation efficacy was greater than 8.5 log. The CFUs of E. coli were reduced by 7.5 log, B. cereus were 8 log; however, PPV and S. aureus cannot be inactivated sufficiently. The activities of factor II, VII, IX, X, XI, XII, fibrinogen, IgG, IgM stayed over 95% compared to untreated, and Factor V and VIII activity was maintained at 46-63% and 77-82%, respectively.” ² Yang C, Bian G, Yang H, Zhang X, Chen L, Wang J (2016) Development of High Hydrostatic Pressure Applied in Pathogen Inactivation for Plasma. PLoS ONE 11(8): e0161775. doi:10.1371/ journal.pone.0161775

### SUMMARY

A system and method to apply high pressure to the product
Containers for the biologic products to be used when applying high pressure to reduce, kill, destroy, eliminate or inactivate unwanted cells, while preserving, maintaining, keeping, protecting the quality, efficacy, function, shape, interfacing features, form, properties of the desired cells, which are words that are synonymous for the purposes of this disclosure specification.

This disclosure includes the following:

1. A system that subjects a health care biologics product, in original or temporary or transitory or consumable or disposable containers (e.g., flow through tubing, tubing segments, vials, pouches, boxes, bottles, bags, balloons, packaging, wraps, sponges), synonymous for the purposes of this disclosure, to high pressure using a working fluid, a pressure vessel and the product container in order to reduce unwanted cells, while preserving an acceptable levels of the desired cells. The disclosure may also include the following:

1.1. A method that
1.1.1. Implements a processing cycle(s) with cycle parameters of.
   1.1.1.1. working fluid pressures,
   1.1.1.2. working fluid water temperatures,
   1.1.1.3. certain ramp rates, waveforms and hold times under these conditions,
   1.1.1.4. subjecting the product to multiple cycles with differing parameters between cycles,
   1.1.1.5. working fluid type
1.1.2. May utilize additives, stabilizers, catalysts, protectants, destabilizers, denaturing agents, which are synonymous for the purposes of this disclosure specification, to preserve the product before, during and after subjecting it to processing.
1.1.3. May utilize pre or post processing methods to improve, facilitate, prime, finalize, compliment the core pressure processing method.

1.2. A specific container that withstands the cycle, with specifications of:
1.2.1. Dimensions, geometry, materials and material properties and methods to adjoin materials, shape materials, form materials.
1.3. The system is scalable to both be small enough to transport into austere environments or large enough to process high volumes of health care products in one batch.

### BRIEF DESCRIPTION OF DRAWINGS

FIG. 1 depicts an erythrocyte (red blood cell) according to various embodiments.

FIG. 2 depicts a pathogen according to various embodiments.

FIG. 3 depicts a blood bag according to various embodiments.

FIG. 4 flow chart for high pressure processing of food.

FIG. 5 depicts a food processing worker loads unprocessed food in its vacuum sealed packaging into a loading cartridge which travels on a conveyor belt into the pressurization machine in the background.

FIG. 6 is a representation of how high pressure processing disrupts the cellular structure and walls of bacteria, specifically.

FIG. 7 is a microscopic picture of a disrupted Listeria (bacteria) cell after high pressure processing.

FIG. 8 illustrates a bag, placed in a high pressure processing pressure chamber, showing the equal and opposite forces acting on the packaging, the working fluid and the walls of the pressure vessel.

FIG. 9 illustrates a possible splitting, peeling or damage to a surface mate between two films related to headspace volume compression and expansion.

FIG. 10 illustrates pooling, or combining, units of product (on the right) into one large mixing bag (on the left), including passing the units through a pre-process filtering step. A post-mixing filtering step may also be used.

FIG. 11 illustrates a typical biologics bag that has multiple inlet and outlet ports.

FIG. 12 shows a container in the form of a bottle.

FIG. 13 depicts a threaded mate between the cap and the main cylindrical or conical or spherical or rectangular body of the container.

FIG. 14 depicts the dimensions necessary for a hermetically sealed container that prevents influx of the high pressure processing working fluid into the container.

### DETAILED DESCRIPTION

### Parameters and Processing

The baseline pressure range that inactivates most pathogens are 50-300 MPa, while preserving the efficacy of plasma, limiting the hemolysis of red blood cells and is on the edge of maintaining platelet stability. The disclosure considers the right settings for these parameters to disrupt the unwanted cells while maintaining efficacy of the desired cells.

This disclosure considers parameters and processes that may affect the viability such as:
Pressure,
   waveforms, for example a sawtooth pattern, an ascending wave, a square-wave, a polynomial application, a sine wave, a rectangular wave, a resonant frequency wave or other pulsing wave forms,
   ramp-up and ramp-down timing,
   hold and relax times,
   number of cycles,
Temperature,
   may not be static, but may be variable during high pressure processing,
Pre-processing, before high pressure processing,
   freeze product, then thaw, with variable freeze and thaw rates prior to processing with pressure.
      by altering these parameters, the ice crystal lattice of the structures may be tuned to preserve the desired cells while disrupting the unwanted cells by making them more vulnerable to high pressure processing.
      the product could be fresh, never frozen in liquid form.
      the product could have been thawed for use, not used, then treated with high pressure processing and frozen afterwards. For example, at a hospital's blood bank, the unit may have been thawed for use, not used by the ordering physician, and then treated with high pressure processing rather than discarded or repurposed due to the expiry of thawed plasma (on the order of a few weeks, when refrigerated).
   preliminarily applying a complimentary method to remove unwanted cells that high pressure processing may not eliminate in the range of pressures applied,
      Leukocyte filtering or other filtering,
      UV light treatment with or without additives,
      solvent detergent treatment,
   Conversion of the product
      the blood could be lyophilized (freeze dried) to preserve structure.
      converted into cryoprecipitate,
   An additive could be present in the container during filling, or added after the container is filled with the blood product. This could be in solid, liquid or gas phases.
      An additive to protect the desired cells or increase the likelihood of the required efficacy.
      An additive to amplify or increase the likelihood of reducing unwanted cells by pre-emptively weakening, disrupting, altering or changing the unwanted cells. For example, a chemical or biologic that would weaken the lipopolysaccharide layer of a bacterial membrane. One analogy is creating a crack in glass, which is propagated during high pressure processing. Another analogy is a diesel engine, which combusts at a pressure point. This disclosure considers a chemical additive or treatment that would destroy the unwanted cells at sufficiently high pressures, while being not so high as to damage the desired cells.
Post-processing, after high pressure processing,
   filtering the product through a membrane or porous filter may be used to
      remove fragments of the damaged desired cells (for example red blood cells due to hemolysis), or
      remove fragments of the damaged unwanted cells (for example, disrupted pathogens)
   a secondary complimentary reduction method to remove unwanted cells that high pressure processing may not eliminate in the range of pressures applied, such as those listed in pre-processing.

### Additives, Stabilizers, Protectants, Filters

This disclosure considers that additives may be used to protect the desired biologic cells (e.g., red blood cells (red blood cells), platelets) and increase the likelihood of disrupting the unwanted cells when subjected to high pressure processing. This disclosure considers the function of these additives to:
Strengthen, make more rigid, protect, preserve, fortify, reinforce, change the material properties or the structure of the desired cells to survive the high pressures.
Increase the elasticity of the desired cells to bend, contort, deform, absorb the high pressures.
To affect a temporary modification (e.g., chemical, structural, composition, thermal, mechanical, radiative) to survive the HPP process and then reverse the alteration after the process with a chemical, a filter or a reversal method.
Damage, deactivate, destabilize, weaken, compromise, disable, make vulnerable the unwanted cells so that they are reduced while the desired cells survive.
Alter, expand, make more favorable, reduce, increase, remove any of the cycle parameters. For example, an additive may reduce the needed pressures for pathogen inactivation.

Each desired cell product may require a bespoke additive or combination of additives. Compounds found in deep sea creatures, such as organic osmolytes, trimethylamine N-oxide (TMAO), squalamine (found in dogfish sharks), Limulus Amebocyte Lysate (found in horseshoe crabs), other amoebocytes, antimicrobial peptides, including peptide fragments of histones, leukocyte lysates, coagulogens, hemocyanin, cold water oxygen (found in crocodile icefish), oxygen infused water or saline, hemerythrin (found in brachiopods), chlorochurion; and compounds used in the de/glycerolization process to freeze red blood cells, such as dimethyl sulfoxide (DMSO) may protect red blood cells and other cells. Additives such as Trehelose, which has been used as a platelet cryoprotectant and in lyophilization (freeze dry); thrombocytes; lymphocytes; or apatite-trehalose-saline may protect the platelets or other cells during high pressure processing.

Other additives that this disclosure considers are thrombin; lactoferrin; collagen and ristocetin; amotosalen; psoralen; hyaluronic acid; glucosamine; monosaccharides and disaccharides; albumin, serum albumin, factor concentrates, immunoglobulin, fibrinogen; proteins such as monoclonal antibodies, peptides, interferons; surface associated glycans, formylated 6 peptides, lipopolysaccharide; saline adenine glucose mannitol; vitamins such as riboflavin, vitamins A, C, D and E; amino acids; antimicrobials such as steroid-polyamine conjugate compounds; antibiotics such as hygromycin, vancomycin and others that cover methicillin-resistant staphylococcus aureus, ceftobiprole and others that cover pseudomonas aeruginosa, linezolid and others that cover vancomycin-resistant Enterococcus; antivirals such as tenofovir, tipranavir, simeprevir, remdesivir, elbasvir; antiparasitic drugs such as artemisinin, atovaquone-proguanil, quinine sulfate with doxycycline, primaquine phosphate; anti-angiogenic factors and agents; enzymes, enzyme inhibitors, proteasomes, proteasome inhibitors; pH buffers, acids or alkalinity increasers; salt, urea, saline, crystalloids, colloids; liposome structures or liposomes, and combinations, of two or more at specific concentrations, or substitutions of these additives.

The disclosure also considers protecting the desired cells (e.g., red blood cell, platelets and other cells) with a gas-impermeable coating or a chemical that prevents gas from entering cell. Being protected, the product could be subjected to high pressure processing. In some embodiments, another chemical or gas could be added to the product, which would attach, infuse or bond to the unwanted cells, which would then increase the likelihood of deactivation during high pressure processing. For example, the unwanted cells would be inactivated by explosive decompression cavitation during high pressure processing. These processes of protection and priming could be used in conjunction or separately.

These additives could be in the form of a solution, added to the container in advance of collection, after collection, before mixing or after mixing. For example, blood products require anti-coagulants in the container, as shown in Error! Reference source not found., and these anti-coagulants may provide a secondary purpose of stabilizing the cells for high pressure processing. The chemical composition of these nutrient solutions may be adjusted to stabilize the cells. The disclosure considers citrate, dextrose, phosphate, adenine, citric acid, citrate-glucose, acidified-citrate-dextrose, citrate-phosphate-dextrose with and without adenine, and combinations of these additives.

### Container/Vessel

The system requires a container that can withstand the high pressures exerted on the container that are translated to the product. The materials, construction, geometry, joints, interfaces, etc. all integrate for a container design that can withstand the process.

### Type and Shape

The disclosure considers multiple embodiments of the container, including a bag made by joining two sheets of film in an enclosed perimeter and a pre-formed container. As shown in Error! Reference source not found., this disclosure considers the need to carefully select materials, dimensions, geometry, adhesion methods, elastic moduli (e.g., Young's, shear, bulk moduli), strain rate for plastic deformation, yield strength, mating of materials with dissimilar material properties in order for the container to compress, compact, deform, disjoint, delaminate, stretch, contort and shift in order for the container to survive high pressure processing. Because high pressure processing applies a balanced sum of forces, containers do not collapse; but, rather, they either elastically or plastically deform. Dissimilar materials will compress at different rates, putting stress on the joints or mating features. The packaging and product will compress at different rates due to their different moduli of elasticity and other mechanical properties.

Bag/Pouch This disclosure considers a bag or pouch made by joining two sheets of film around the perimeter with a seam (e.g., RF weld, ultrasonic weld, thermal process (melting and matrix solidification), chemical adhesion, mechanical adhesion, other joining of the two films).
Pre formed Container This disclosure considers a preformed container such as a bottle, box, pouch that is formed into a shape capable of containing a liquid. This container may be made through blow-molding, injection molding, thermoforming, 3D printing. The container may require a cap, such as the threaded caps shown in Error! Reference source not found., Error! Reference source not found. and Error! Reference source not found. The cap or closure, such as a clamshell, may be threaded, may have and interference fit, may have a specific gap fit, may be adhered to the rim of the cylindrical body by means of radiofrequency welding, ultrasonic welding, chemical adhesion or other mechanical locking features that would close the container and make it impermeable to the working fluid used to transfer pressure during high pressure processing. The cap may be made of a membrane material that allows communication of certain substances while preventing the ingress of the working fluid.

### Physical Properties

This disclosure considers the design selection of elastic moduli (e.g., Young's, shear, bulk moduli), viscosity, creep rate, durometer, compatibility with sterilization methods (e.g., gamma radiation, ethylene tri-oxide, autoclave), surface energy, flexibility, stiffness, Poisson's ratio, shear strength, surface roughness, yield strength, biocompatibility, non-pyrogenicity, hemocompatibility, non-genotoxicity, non-cytotoxicity, non-irritation, non-toxicity, non-leachable, non-extractable, opacity, thermal expansion, glass transition temperatures, melting point, coefficient of thermal expansion.

### Capacity and Total Volume

Pooling, as seen in Error! Reference source not found., is beneficial because the resulting pool has a near-average biological profile. For example, blood plasma consists of coagulation factors. Each donated unit, typically about 250 mL in volume, has differing levels of these factors. When pooling, a unit with lower than average factors may be mixed with a unit with higher than average factors, which would bring the pooled product factor level closer to the average of the donor population. The large pooling bags, which can range in size from, for example, 500 mL to 5 L to 10 L and more, can be processed using high pressure processing and then redistributed into smaller transfusable units. In some embodiments, after using the pooling bag, the mixed product could then be distributed into smaller transfusable units, and then these individual units could be processed using high pressure processing. This pooling process with HPP processing could be used on any biologic that is in liquid form and could be transferred between containers.

Total volume of the sample may range from a few milliliters to several liters.

### Ports

The disclosure considers that the container may be filled with product and then after treatment, that the treated product can be transfused or administered to a patient. As such, there may be an inlet port and an outlet port, which may be the same port. There may be additional ports or connectors to inject substances into the container. These ports and/or caps and the interface with the main container must withstand the high pressures. As shown in Error! Reference source not found., these ports are typically tubular in cross-section in order to access the internal components of the bag. These ports are mated to the bag, typically inserted between layers of film. Due to different material properties, dimensions and geometry, these ports and the junction with the bag will strain and deform at different rates than the main body of the bag, potentially leading to a failure of the packaging.

### Head-space/geometry

This disclosure considers predicable levels of compression of the container (solid) and the biologic (typically liquid, semi-liquid, solid and gas mixtures). There may be a non-zero amount of gas or non-product fluid in the container, called the headspace, as shown in Error! Reference source not found.. Specifying headspace, per the container design and the cycle pressures and other dynamics is critical to the container surviving the high pressure processing because the product and the headspace may have different moduli of elasticity (and yield strength), which results in one material compressing and deforming (elastically or plastically) at a greater or different strain rate than the headspace. The headspace, which may include a gas, will compress more than the solid container and the product will compress, which would stress joints or mating features of the container. This volume has to be such that there isn't too much headspace, the amount of gas in the container, which would result in putting stress on the seams or mating interfaces due to the difference in compression between gases, liquids and solids. If there is not enough headspace, the container may compress more than the liquid, which would put internal stresses on the seams or mating interface of the container, possibly resulting in rupture and leakage.

The volume or quantity of headspace could be measured via a flowrate meter, a scale or another indicator fixture to meter or measure the target volume or mass. There may be markings on the container to indicate the proper level of headspace. There may be a supporting machine or mechanism to add the inert or other gas to the container with or without a filtration method.

### DETAILED DESCRIPTION OF DRAWINGS

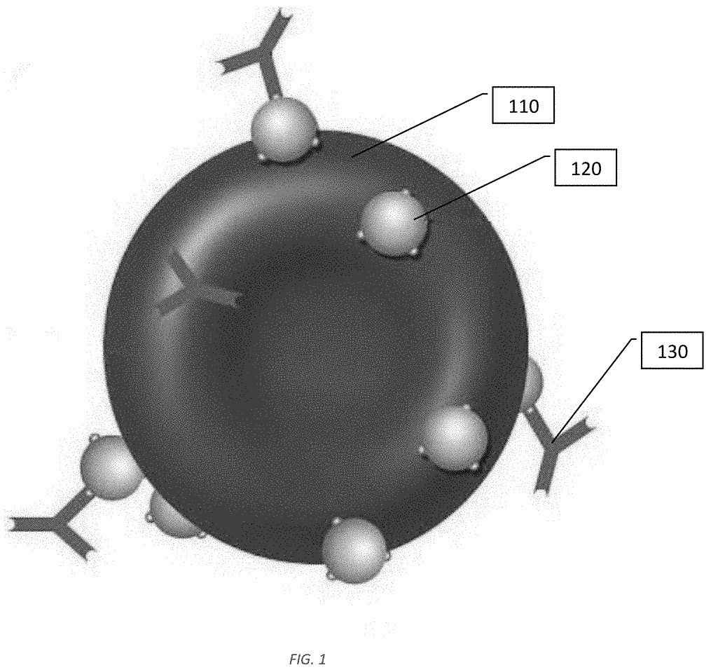

FIG. 1 depicts an erythrocyte, commonly known as a red blood cell, with antigens 120 and antibodies 130. An additive could be designed to bond, react with or attach to an antigen to either remain on the exterior of the cell or to be transmitted into the cell via the antigen. Similarly, an additive could be designed to bond or attach to the antibody to transport it and attach to the antigen. Additives could be designed to coat or bond with the membrane 110 of any type of cell.

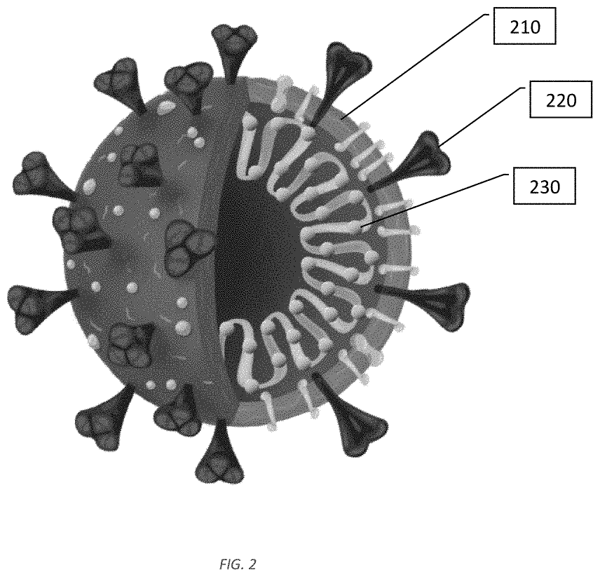

FIG. 2 depicts a pathogen with a membrane 210, a spike protein 220 and the enclosed RNA 220. A catalyst or destabilizing agent could be designed so as to bond or attach to the membrane or proteins in order to disrupt the structure of the pathogen or unwanted cell at pressures, temperatures, frequencies that do not affect the desired cells.

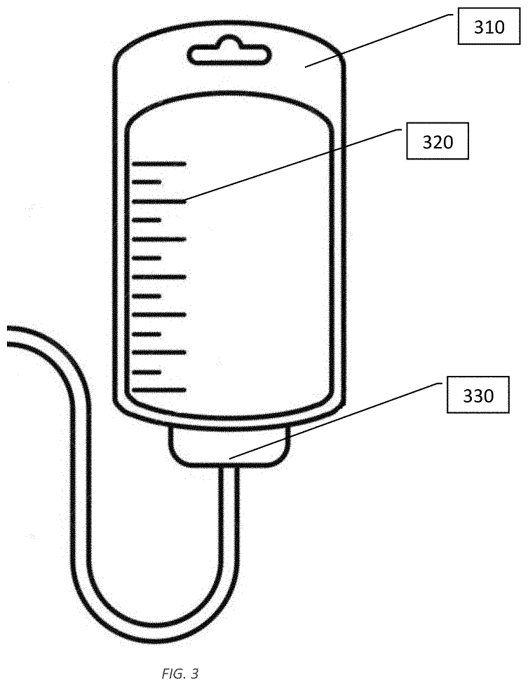

FIG. 3 depicts a blood bag with made by welding, fusing or adhering two layers of film 310, including a port 330, which is pressed between the two layers of film. The headspace of the liquid in the container could be measured by adding graduation markers 320.

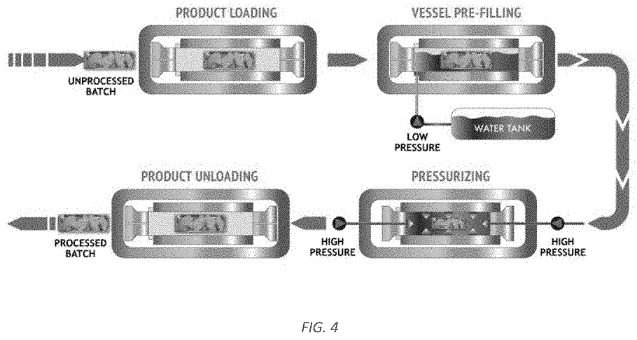

FIG. 4 flow chart for high pressure processing of food. The unprocessed batch, in its final packaging (e.g., bottled juice, vacuum plastic wrapped meat) is loaded into a dry, empty pressure vessel. The pressure vessel is securely closed and then flooded with water, typically near freezing temperatures. The pressure is increased to the target pressure, typically holding at the target pressure for a specific amount of time. This disclosure considers increasing then reducing pressure, or cycling pressure in many different manners. After the total cycle has completed, the water is evacuated from the chamber and the processed product batch is recovered and allowed to dry, and typically put into a refrigerator or freezer.

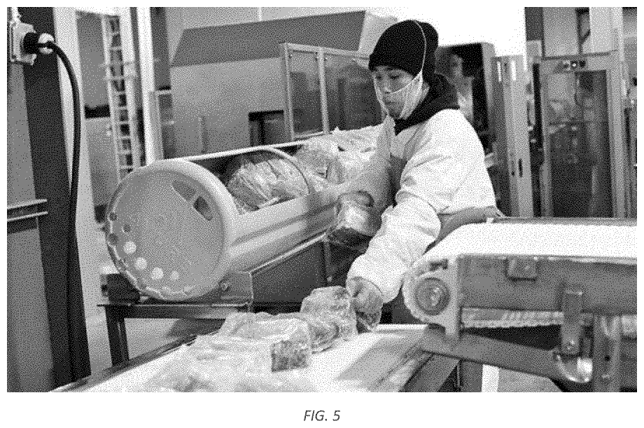

FIG. 5 depicts a food processing worker loads unprocessed food in its vacuum sealed packaging into a loading cartridge which travels on a conveyor belt into the pressurization machine in the background. This disclosure considers pre or post processing to compliment the pressurization. Additionally, this disclosure considers adding catalysts, additives or other chemicals to the interior of the packing prior to pressurization.

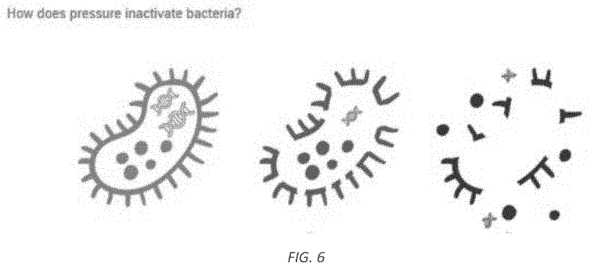

FIG. 6 is a representation of how high pressure processing disrupts the cellular structure and walls of bacteria, specifically. This disclosure considers methods for preserving the desired cells, e.g., red blood cells, platelets, while disrupting the unwanted cells by strengthening the desired biologic cells, weakening the unwanted contaminant cells, using a method that targets the unwanted cells or by utilizing other methods described by this specification.

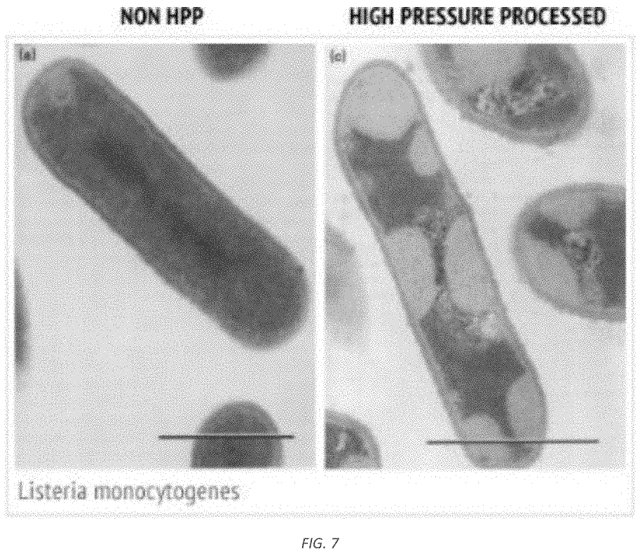

FIG. 7 is a microscopic picture of a disrupted Listeria (bacteria) cell after high pressure processing. This disclosure considers catalysts that would allow the unwanted cells' structure to be weakened so that they were disrupted at lower pressures, temperatures or via different cycles than the wanted cells were affected by.

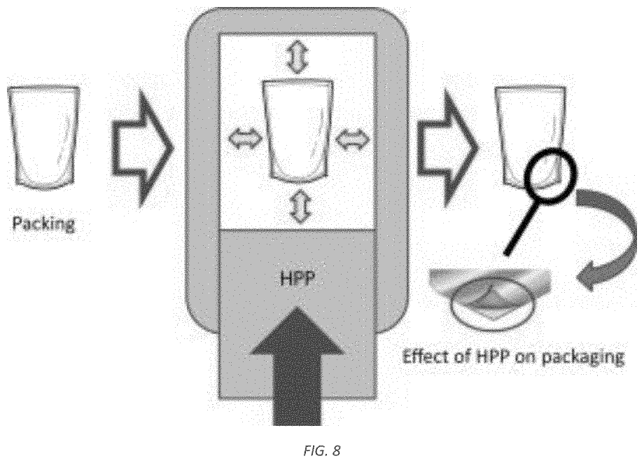

FIG. 8 illustrates a bag, placed in a high pressure processing pressure chamber, showing the equal and opposite forces acting on the packaging, the working fluid and the walls of the pressure vessel. This illustrates the concept of the summed balance of forces being applied to the packaging where the packaging and product will not implode as long as the force can be transferred through deflection, compression, deformation, elongation through the container, through the product and conversely through the other side of the container. Also, within the packaging there are variable and different moduli of elasticity and strain rates due to geometry (e.g., the corners of the seam experiencing different angles of stress), material properties (including the seam which may have different properties than either of the independent film materials), dimensions (e.g., the long edge may experience more strain than the shorter edge), viscosity of the product, parameters of the cycle (e.g., a quick increase in pressure may create more strain). The illustration shows the potential damaging effects of high pressure processing on container packages, specifically the peeling of a seam of film layers welded or adhered together to make a pouch or bag. This disclosure considers different materials, manufacturing methods, dimensions, adhesives and adhesion techniques such that the container withstands the pressurization.

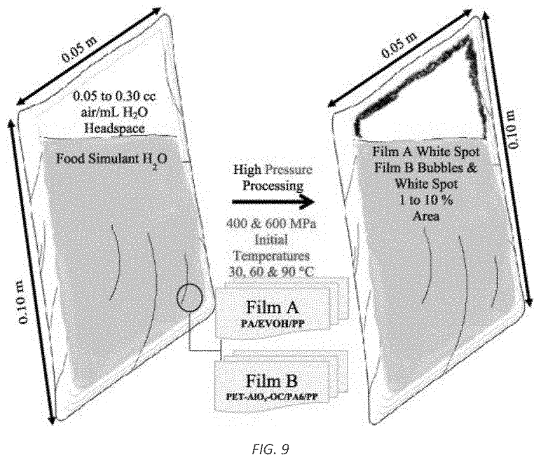

FIG. 9 illustrates a possible splitting, peeling or damage to a surface mate between two films related to headspace volume compression and expansion. The disclosure considers the need to specify, meter or control a headspace, or amount of space of gas or non-product fluid in the enclosed container. This figure shows a possible splitting, peeling or damage to a surface mate between two films related to headspace volume compression and expansion.

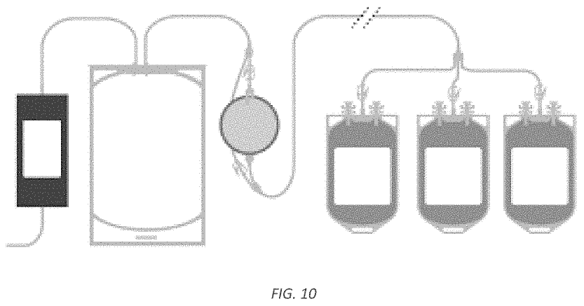

FIG. 10 This disclosure considers pooling, or combining, units of product (on the right) into one large mixing bag (on the left), including passing the units through a pre-process filtering step. This disclosure also considers post-mixing filtering step.

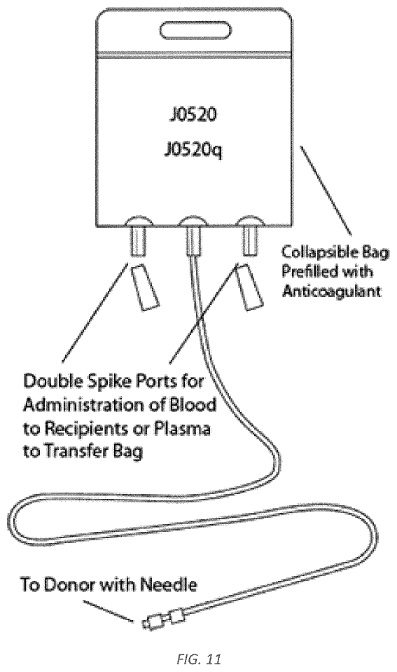

FIG. 11 illustrates a typical biologics bag that has multiple inlet and outlet ports. Additionally, this illustration shows the addition of anticoagulant chemicals into the bag prior to the donated blood being transferred into the bag. This disclosure considers the addition of such anticoagulants as well as other stabilizers, preservatives, protectants, catalysts that are needed to ensure that the efficacy of the product is maintained and that the pathogen kill level is sufficient.

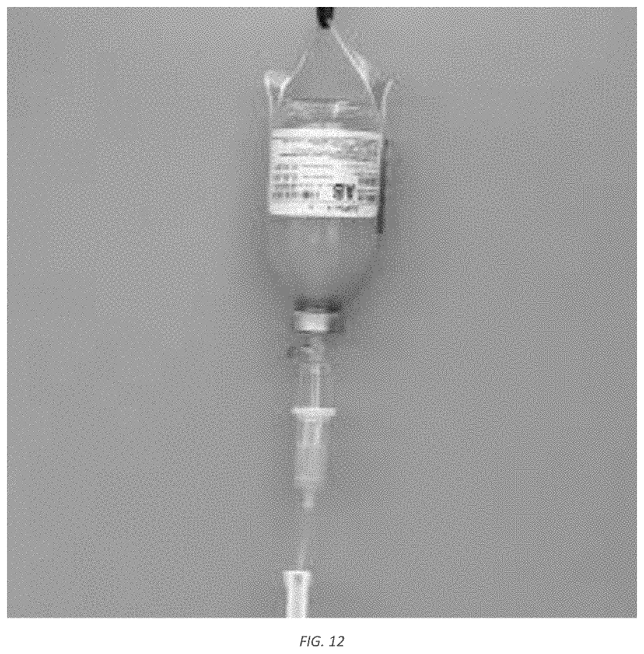

FIG. 12 shows a container in the form of a bottle. This disclosure considers a cylindrical container, similar to food packaging for juices and other fluids, with a cap that has the necessary ports for collection and administration of a biologic.

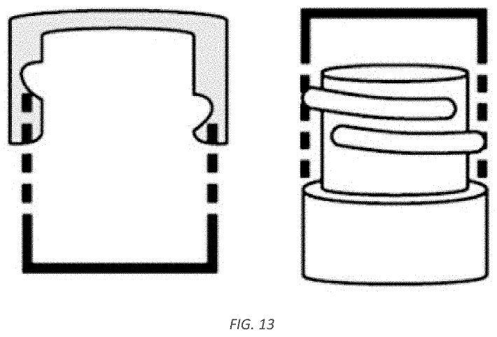

FIG. 13 depicts a threaded mate between the cap and the main cylindrical or conical or spherical or rectangular body of the container. Under pressure the threads of the cap are forced against the thread of the body. This disclosure considers hermetically sealing these threads via welding the threads together or by welding a cap onto a top with specific gaps or interference fits.

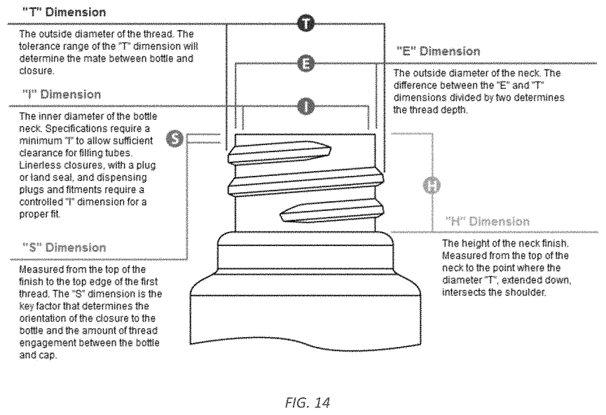

FIG. 14 this disclosure considers the dimensions necessary for a hermetically sealed container that prevents influx of the high pressure processing working fluid into the container.

What is claimed is:

1. A neutralization method comprising the steps of:
adding a cellular stabilizing agent to a biological matrix to preserve a predetermined level of desired cells; and
subjecting the biological matrix to pressures above 5,000 psi, while maintaining the temperature of the material at 4° C. or lower.

2. The method of claim 1 wherein one or more of parameters of pressure, temperature, and time is varied in one or more of a waveform, sawtooth pattern, an ascending waveform, a square-waveform, a polynomial waveform, a sine waveform, a rectangular waveform, a resonant frequency waveform, pulsing waveforms, and hold and relax times.

3. The method of claim 1 wherein the method further comprises
a pre-process or post-process including of one or more of freezing or thawing the material with variable freeze and thaw rates, Leukocyte filtering, porous filtering, membrane filtering, UV light treatment with or without additives, solvent detergent treatments, lyophilization, and cryoprecipitate conversion.

4. The method of claim 1 wherein the stabilizing agent includes one or more of additives, stabilizers, catalysts, protectants, destabilizers, and denaturing agents.

5. The method of claim 4, wherein one or more of the additives, the stabilizers, the catalysts, the protectants, the destabilizers, and the denaturing agents includes or more or of specific concentrations or proportions of organic osmolytes, trimethylamine N-oxide (TMAO), squalamine, Limulus Amebocyte Lysate, other amoebocytes, antimicrobial peptides, including peptide fragments of histones, leukocyte lysates, coagulogens, hemocyanin, cold water oxygen, oxygen infused water or saline, hemerythrin, chlorochurion, dimethyl sulfoxide (DMSO), trehelose, thrombocytes, lymphocytes, apatite-trehalose-saline, thrombin, lactoferrin, collagen, ristocetin, amotosalen, psoralen, hyaluronic acid, glucosamine, monosaccharides, disaccharides, albumin, serum albumin, factor concentrates, immunoglobulin, fibrinogen, proteins, monoclonal antibodies, peptides, interferons, surface associated glycans, formylated 6 peptides, lipopolysaccharide, saline adenine glucose mannitol, riboflavin, vitamins A, C, D and E, amino acids, antimicrobials, steroid-polyamine conjugate compounds, antibiotics, hygromycin, vancomycin, ceftobiprole, antivirals, tenofovir, tipranavir, simeprevir, remdesivir, elbasvir, antiparasitic drugs, artemisinin, atovaquone-proguanil, quinine sulfate with doxycycline, primaquine phosphate, anti-angiogenic factors and agents, enzymes, enzyme inhibitors, proteasomes, proteasome inhibitors, pH buffers, acids, alkalinity increasers, salt, urea, saline, crystalloids, colloids, liposome structures or liposomes.

6. The method of claim 4, wherein the additives, stabilizers, catalysts, protectants, destabilizers, denaturing agents are added prior to, during or after the material is subjected to the neutralization method.

7. The method of claim 1, wherein a gas-impermeable coating or chemical shell is applied to the desired cells to withstand the neutralization method.

8. The method of claim 1, wherein a catalyst, destabilizing agent or electrical charge is applied to the biologic matrix to assist, compliment, amplify the effects of the neutralization of the unwanted cells. The method of claim 1, wherein the method is applied to flexible packaging of specific dimensions, form, geometry, materials and material properties, and made using specific methods to form a container that transmits pressure without rupture.

9. The method of claim 8, wherein the additives, stabilizers, catalysts, protectants, destabilizers, denaturing agents are packaged in the container prior to the biologic material being added to the container.

10. The method of claim 1, further comprising a headspace including one or a combination of ambient air, inert gas, CO2, the volume of which is metered, controlled or measurable.

11. The method of claim 1, wherein the method reduces unwanted cells in a biologic matrix.
